(19) United States
(12) Patent Application Publication
(10) Pub. No.: US 2016/0174813 A1
(43) Pub. Date: Jun. 23, 2016

(54) ENDOSCOPE AND ENDOSCOPE TIP

(71) Applicant: OLYMPUS WINTER & IBE GMBH, Hamburg (DE)

(72) Inventors: Jens ROSE, Hamburg (DE); Uwe SCHOELER, Hoisdorf (DE)

(73) Assignee: OLYMPUS WINTER & IBE GMBH, Hamburg (DE)

(21) Appl. No.: 15/058,504

(22) Filed: Mar. 2, 2016

Related U.S. Application Data
(63) Continuation of application No. PCT/EP2014/002279, filed on Aug. 20, 2014.

(30) Foreign Application Priority Data
Sep. 3, 2013 (DE) 102013217500.0

(51) Int. Cl. A61B 1/00; A61B 1/07; A61B 1/04; A61B 1/06

(52) U.S. Cl. CPC A61B 1/0011; A61B 1/0676; A61B 1/00096; A61B 1/07; A61B 1/04

(57) ABSTRACT

An endoscope including: an endoscope tip arranged at the distal end of the endoscope. The endoscope tip including: an outer wall having an opening; and a window hermetically joined into the opening, the window being at least partially optically transparent; wherein the outer wall has a ceramic material.

### CROSS-REFERENCE TO RELATED APPLICATION

The present application is a continuation of PCT/EP2014/002279 filed on Aug. 20, 2014, which is based upon and claims the benefit to DE 10 2013 217 500.0 filed on Sep. 3, 2013, the entire contents of each of which are incorporated herein by reference.

### BACKGROUND

1. Field

The present application relates to an endoscope having an endoscope tip arranged at the distal end of the endoscope, the endoscope tip comprising an outer wall having an opening and an optically at least partially transparent window joined hermetically into the opening.

The present application further relates to an endoscope tip for an endoscope, wherein the endoscope tip comprises a tip body having an outer wall that has an opening and an optically at least partially transparent window joined hermetically into the opening.

2. Prior Art

In the prior art, video endoscopes for surgical use are known which have an optically transparent inspection window at the distal end of a shaft. To protect mechanical, electrical and/or optical components of the video endoscope within the shaft, or respectively behind the inspection window, in particular when cleaning, disinfecting and/or autoclaving, the inspection window must be hermetically inserted or joined in a corresponding opening in the distal end of the shaft. This is generally accomplished by means of a soldered connection.

At the same time, the outer surfaces of the video endoscope must consist of biocompatible materials in order to avoid complications in the surgical use of the video endoscopes, for example from tissue irritations or allergic reactions. For this reason, the shaft of known video endoscopes generally consists of stainless steel, such as stainless steel 1.4301, and the inspection window consists of sapphire or another corundum.

In this context, the problem results that the thermal expansion coefficients of sapphire of approximately 6×10⁻⁶/K and stainless steel 1.4301 of approximately 16×10⁻⁶/K strongly differ from each other. Consequently, stress arises in the glass during soldering and/or the subsequent cooling phase which can cause the glass to break.

To avoid this problem, EP 2 263 518 A1 proposes an endoscope which comprises a window on a distal end of the endoscope, an inner body with a first material on the distal end, and a jacket with a second material on the distal end, wherein the window and the inner body are joined. In this manner, the inner body can be selected from a material without consideration of biocompatibility with a thermal expansion coefficient that lies close to the thermal expansion coefficient of the window. For example, NiFe alloys with a nickel mass fraction of approximately 42%, or NiCoFe alloys with a nickel mass fraction of approximately 29% and a cobalt mass fraction of approximately 17% are proposed for this. The biocompatibility is ensured in that the inner body is covered with the jacket and thereby does not form an outer surface of the endoscope.

The construction of the corresponding endoscope is, however, complex and hence potentially error-prone. In particular, an additional seal is needed between the jacket and window in order to ensure autoclavability.

### SUMMARY

Based on this prior art, an object is to provide an alternative solution for an endoscope with an endoscope tip having a window, wherein defects such as breaking the window from thermally-induced stress is avoided, and the construction is kept as simple as possible.

Such object can be achieved with an endoscope having an endoscope tip arranged at the distal end of the endoscope, the endoscope tip comprising an outer wall having an opening and an optically at least partially transparent window joined hermetically into the opening, wherein the endoscope is developed in that the outer wall has a ceramic material. The endoscope tip at the distal end can consist of a ceramic material which can have a thermal expansion coefficient within the range of the thermal expansion coefficient of sapphire.

An advantage of such configuration is that ceramic material and transparent inorganic or mineral material such as glass or sapphire glass have comparable thermal expansion coefficients. Because a material of the outer wall surrounding the window joined in the opening in the outer wall has a thermal expansion coefficient comparable to the window, or a material of the window, thermally induced stress in the window is generally reduced or excluded. This is possible over a large temperature range, such as the temperatures arising during autoclaving or soldering.

Another advantage is that ceramic material reacts in a chemically inert manner and is therefore generally biocompatible. Furthermore, ceramic surfaces, or respectively surfaces of ceramic material, can be treated or sealed in many ways, such as by means of coating methods or glazing methods. For this reason, an outer surface of the outer wall of the endoscope tip can be advantageously easy to adapt to the properties desired in an individual instance, such as with regard to the appearance, surface structure, and/or resilience.

Within the context of this disclosure, an endoscope tip is understood to be the part of an endoscope that is arranged at a distal end of a shaft of the endoscope. The endoscope tip can comprise an area or section of the shaft at its distal end, or is connected to a distal end of the shaft.

The endoscope can be configured as an optical endoscope and/or a video endoscope. For this purpose, at least one optical component can be provided or arranged in a hermetic chamber of the endoscope surrounded by the window and the outer wall. An optical component can be a prism, a mirror and/or an electronic imaging device, such as a photosensitive semiconductor chip, such as a CCD sensor. The at least one optical component can enable direct or indirect observation of surroundings of the endoscope tip from a proximal end of the endoscope, such as opposite the endoscope tip.

The window can be joined into the opening in the outer wall by means of a soldered connection. To this end, the window and the outer wall can be metalized or coated with metal on surfaces to be joined to each other respectively, and can then be soldered together at the metalized or coated surfaces using a soldering material. This method is also termed soft soldering. In this context, a material for metalizing or coating, and/or a soldering material, can comprise a precious metal, such as silver or gold. This ensures that the soldered connection between the window and outer wall is formed corrosion-resistant and biocompatible. Alternatively, these surfaces can be connected to each other by active soldering, which can be without prior metalization as well.

A window material can be sapphire or sapphire glass. Sapphire has the necessary optical properties for an endoscope, such as a video endoscope, with regard to transparency and reflection to visible light, and it is biocompatible.

The window can have a curved shape, such as being curved to the outside.

This renders a particularly large area or section of the surroundings at the endoscope tip optically discernible by means of at least one optical component behind the endoscope tip. There can be a large range of different observation or viewing angles with a constant glass thickness or window thickness in the line of sight. This renders the endoscope suitable for video endoscopes with a variable or changeable line of sight.

Furthermore, the ceramic material can be silicon nitride, zirconium oxide, alumina-toughened zirconia (ATZ), zirconia-toughened alumina (ZTA), aluminum oxide or strontium titanate. Silicon nitrite with the molecular formula Si₃N₄ and strontium titanate with the molecular formula SrTiO₃ are ceramic materials whose mechanical properties also permit the use in areas of an endoscope which can be subject to mechanical stress. Aluminum oxide in this regard has the molecular formula A₂O₃. Zirconium oxide can be zirconium dioxide, i.e. ZrO₂. This makes it possible to design the endoscope tip of an endoscope as a shaft, or at least part of a shaft of the endoscope. The window can consists of sapphire.

The endoscope can have at least one fiber-optic conductor to conduct light from a light source to the endoscope tip.

The light source can be an internal light source of the endoscope, or an external light source, such as being connected or connectable to the proximal end of the endoscope.

The fiber-optic conductor can have a light exit surface which is arranged on the window. This brings about an illumination of the surroundings of the window, and hence an area in front of the endoscope tip observable by means of a lens system of the endoscope.

Also provided is an endoscope tip for an endoscope, such as for an optical endoscope and/or a video endoscope, wherein the endoscope tip comprises a tip body having an outer wall that has an opening and an optically at least partially transparent window joined hermetically into the opening, wherein the endoscope tip is developed in that the tip body, such as the outer wall of the tip body, has a ceramic material. The tip body can consist of a ceramic material.

The endoscope tip can be suitable for an endoscope with the above-described characteristics.

The tip body can be configured as a shaft for an endoscope.

In an alternative embodiment, the tip body can be joinable to a shaft of an endoscope, such as being solderable, wherein the shaft can consist of stainless steel. Ceramic material is generally more break-proof than transparent, inorganic and/or mineral material, such as glass or sapphire. Consequently, thermally induced stress at a connection between the tip body and a shaft, such as a shaft consisting of stainless steel, such as stainless steel 1.4301, is uncritical. Accordingly, a conventional soldered connection can be used or employed at this location regardless of a differing thermal expansion coefficient which may exist between the tip body and shaft.

Further characteristics will become apparent from the description of embodiments together with the claims and the included drawings. Embodiments can fulfill individual characteristics or a combination of several characteristics.

### BRIEF DESCRIPTION OF THE DRAWINGS

The embodiments are described below, without restricting the general idea of the invention, using exemplary embodiments with reference to the drawings, whereby we expressly refer to the drawings with regard to all details that are not explained in greater detail in the text. In the figures:

FIG. 1 schematically illustrates an endoscope according to an embodiment;

FIG. 2 schematically illustrates an endoscope tip according to an embodiment; and

FIG. 3 schematically illustrates an endoscope tip according to another embodiment.

In the drawings, the same or similar elements and/or parts are provided with the same reference numbers in order to prevent the item from needing to be reintroduced.

### DETAILED DESCRIPTION

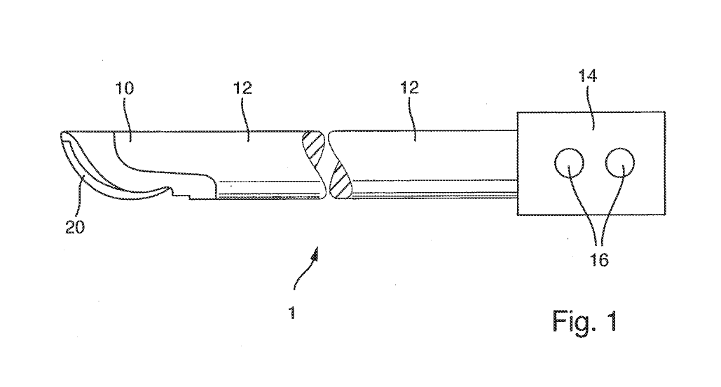

FIG. 1 schematically illustrates a video endoscope 1 with a shaft 12. The shaft 12 connects an endoscope tip 10 arranged on the distal end of the shaft 12 to a proximal area 14 of the video endoscope 1.

The video endoscope 1 can be configured to be inserted with the endoscope tip 10 into a natural or surgically-created bodily cavity in a human or animal body in the context of a surgery in order to perform a visual examination at that location. For this purpose, optical components such as a mirror, prism or lens are provided in the endoscope tip 10 behind a sapphire window 20 in order to capture, or respectively transmit, a picture of the surroundings of the endoscope tip 10. In addition or alternatively, a camera or a CCD chip can also be provided to record a corresponding picture. Mechanical, electrical or mechatronic components, such as to pivot a mirror, or electrical components, such as a photosensitive semiconductor chip to digitally capture the image of the surroundings, can also be provided.

In the proximal area 14 of the video endoscope 1, optical and/or electrical connections 16 can be provided which are connected to the internal components of the video endoscope 1 and can serve to supply voltage or current and read out digital image signals.

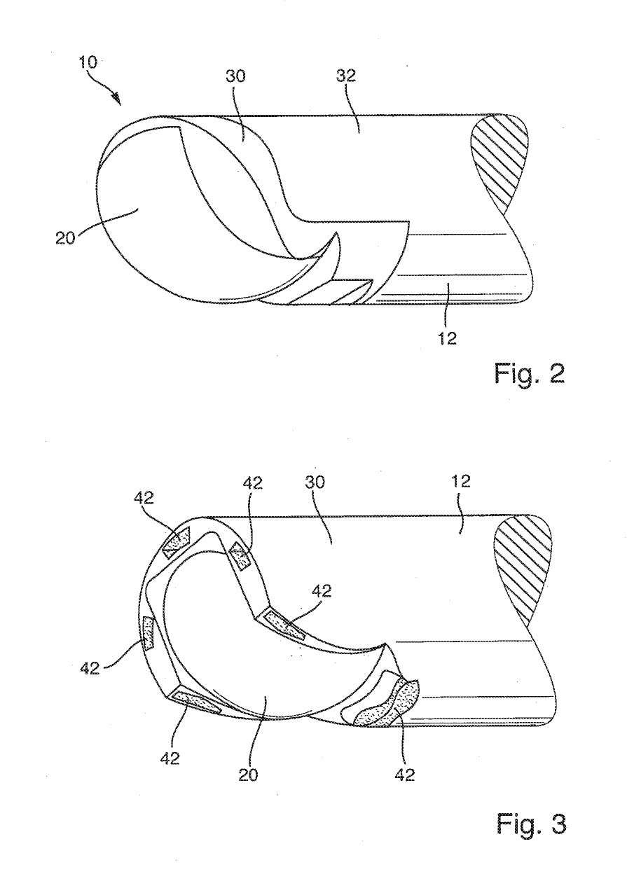

FIG. 2 shows a schematic, enlarged representation of the endoscope tip 10. The endoscope tip 10 comprises a ceramic body 30 with an opening in which a sapphire window 20 is soldered. The ceramic body 30 with the sapphire window 20 is hermetically connected, such as soldered, to the distal shaft end 32 of the shaft 12.

FIG. 3 schematically portrays an endoscope tip 10 according to another exemplary embodiment. In this case, the ceramic body 30 is configured as a shaft 12 for a video endoscope 1. Accordingly, the shaft 12 can be a ceramic body 30 with an opening in which a window 20, which can consist of sapphire, is inserted.

Furthermore, an outer wall of the ceramic body 30 has longitudinally axial channels, which can be aligned along a longitudinal extension of the shaft 12 in which optical fibers or fiber-optic conductors can be embedded. The fiber-optic conductors can be connectable by means of one of the connections 16, which can be an optical connection, to an external light source.

At the endoscope tip of FIG. 3, the optical fibers have light exit surfaces 42, wherein a plurality of light exit surfaces 42 are arranged around the window 20. This ensures an even illumination of an examined area, or surroundings in front of the window 20 for performing an intended examination with the video endoscope.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE NUMBERS

1 Video endoscope
10 Endo scope tip
12 Shaft
14 Proximal area
16 Connection
15 Sapphire window
30 Ceramic body
32 Tube
42 Light exit opening

What is claimed is:

1. An endoscope comprising:
an endoscope tip arranged at the distal end of the endoscope, the endoscope tip comprising:
an outer wall having an opening; and
a window hermetically joined into the opening, the window being at least partially optically transparent;
wherein the outer wall has a ceramic material.

2. The endoscope of claim 1, wherein at least a portion of the outer wall joined to the window is the ceramic material.

3. The endoscope according to claim 1, wherein the endoscope is configured as one of an optical endoscope and video endoscope.

4. The endoscope according to claim 1, wherein the window is joined into the opening in the outer wall by a soldered connection.

5. The endoscope according to claim 1, wherein a material of the window is sapphire.

6. The endoscope according to claim 1, wherein the window has a curved shape.

7. The endoscope of claim 6, wherein the curved shape is a concavely curved shape.

8. The endoscope according to claim 1, wherein the ceramic material is selected from a group consisting of silicon nitride, zirconium oxide, alumina-toughened zirconia (ATZ), zirconia-toughened alumina (ZTA), aluminum oxide and strontium titanate.

9. The endoscope according to claim 1, further comprising at least one optical fiber conductor for conducting light from a light source to the endoscope tip.

10. The endoscope of claim 9, wherein the at least one optical fiber conductor has a light exit surface arranged adjacent to the window.

11. An endoscope tip for an endoscope the endoscope tip comprising:
a tip body having an outer wall that has an opening; and
a window hermetically joined into the opening, the window being at least partially optically transparent;
wherein the tip body has a ceramic material.

12. The endoscope tip of claim 11, wherein the outer wall of the tip body has the ceramic material.

13. The endoscope tip of claim 12, wherein at least a portion of the outer wall joined to the window is the ceramic material.

14. The endoscope tip according to claim 11, wherein the window is joined into the opening in the outer wall by a soldered connection.

15. The endoscope tip according to claim 11, wherein a material of the window is sapphire.

16. The endoscope tip according to claim 11, wherein the window has a curved shape.

17. The endoscope tip of claim 16, wherein the curved shape is a concavely curved shape.

18. The endoscope tip according to claim 11, wherein the ceramic material is selected from a group consisting of silicon nitride, zirconium oxide, alumina-toughened zirconia (ATZ), zirconia-toughened alumina (ZTA), aluminum oxide and strontium titanate.
